(19) United States
(12) Patent Application Publication
(10) Pub. No.: US 2007/0078291 A1
(43) Pub. Date: Apr. 5, 2007

(54) Needle assembly with enhanced steerability

(75) Inventors: Richard A. Terwilliger, Venice, CA (US); L. Michael Cutrer, Chatsworth, CA (US)

Correspondence Address: MCDERMOTT WILL & EMERY LLP, 2049 CENTURY PARK EAST, 34TH FLOOR, LOS ANGELES, CA, 90067-3208, US

(73) Assignee: North American Scientific

(21) Appl. No.: 11/241,173

(22) Filed: Sep. 30, 2005

(51) Int. Cl. A61M 36/00; A61N 5/00

(52) U.S. Cl. 600/7

(57) ABSTRACT

An apparatus for implanting an element within a tissue a cannula having a sharpened tip at a beveled distal end, and a stylet. The stylet is configured to engage the element within a hollow bore of the cannula, so that the element is pushed through the hollow bore out of the beveled distal end onto the tissue, when the cannula is moved relative to the stylet along an axis. A bevel-shaped insert member has a bevel angle that matches the bevel angle of the cannula distal end. The outer surface of the insert member supports the tissue, to prevent the tissue from prolapsing into the interior of the beveled distal end during penetration of the tissue by the sharpened tip. A protruding structure extends from the outer surface of the member, and frictionally holds the member in place along the interior surface of the hollow bore during the penetration.

### BACKGROUND

As an alternative to general surgery, a pattern of radioactive sources may be placed in the body, to treat cancer by destroying cancer cells with low dose radiation.

These radioactive sources or “seeds” may be placed into the body using multiple hollow needles or needle assemblies. The needles may act as holders and carriers of such seeds, until the needles are inserted into predetermined areas of the body. Once the needles are positioned, the seeds may be deployed from each hollow needle by pulling back the hollow needle over the solid wire stylet to permanently place the seeds in the body as the radioactive dose decays over the treatment time. As many as 25 or more needles may be used in each procedure. Typically, a physician may have to prepare the needles or needle and load the seed sources and spacers into each needle prior to the procedure. Bone wax has been used to close the end of the needle. The wax may be placed into the first 2-5 mm of the distal tip of the needle to prevent the radioactive “seeds” from dislodging or falling out prior to insertion of the needle into the body. The doctor may then insert the needles into the patient and deploys the seeds into the area to be treated.

These needle assemblies may include a needle with a sharpened distal tip and an inner solid wire stylet that is used to hold the radioactive seeds in place as the outer cannula is withdrawn thus expelling the seeds into the body at precise locations. The proximal end of the needle may consist of a plastic or metal hub that allows the loading of the radioactive seeds into the needle. The proximal end of the stylet is a plastic or metal handle for manipulation of the stylet.

These devices may be prepared for use by plugging the hollow bore of the needle with a soft or firm material. This material may extend into the first 2-5 mm of the needle bore and contains the seeds to prevent the seeds from falling out before deployment in the body. The needle may be loaded with radioactive seeds held apart by short non-radioactive spacers that position the seeds in the body to achieve an even distribution of radiation to treat the suspected cancer in vivo.

Prior to insertion, the stylet may be axially introduced into the proximal end of the needle and rests upon the stack of seeds and spacers, which are held in place by the material in the bore of the needle. Once the needle is inserted into the body to the proper position, the stylet may be held firm and the needle may be axially moved toward the proximal end of the stylet. This motion deposits the radioactive seeds and spacers into the body in a track or line of seeds as the needle is pulled back.

There may be two principal types of radioactive seeds. The first type may be “free” seeds. “Free” seeds may be individual radioactive seeds that are loaded in the needle with small cylindrical spacers stacked in between the radioactive seeds. The second type is a pre-manufactured “strand” of radioactive seeds that are encapsulated in a biodegradable material that spaces the radioactive seeds apart from one another.

The needles in a brachytherapy procedure may be placed through holes in a grid block that identifies the X-Y position of the needles entering the tissue. The grid block may be sufficiently thick to start the needle into the tissue on a straight path. The needle will encounter different densities of tissue as the needle is advanced to the treatment site. These density changes deflect the straight path of the needle and the user must attempt to compensate for the needle drifting off the intended path. The grid block prevents the needle from being angled into the tissue at the entry site so the user takes advantage of the angled bevel of the needle to attempt to “steer” the needle by rotating it while moving it forward. The angle of the bevel will move the needle tip away from the angle as it is pushed into the tissue, by rotation the needle tip it can be made to move up or down left or right to some degree. Since to needle's bevel angled tip is hollow only the edges of the bevel contribute to the deflection of the needle path and are inadequate to accomplish any large measurable steerage.

The use of bone wax or other materials that are used to plug the needles, and their inability to assist in steering the needle tip to its desired site, may cause complications.

Bone wax may be an inadequate material in the distal end of the needle, even though it creates a dam in the end of the needle, because the material is soft and is prone to being pushed up the needle as the needle is introduced into the body providing little help in steering.

Other types of needle plugs include synthetic plugs. Synthetic plugs are generally square ended, and are positioned at the back of the beveled tip opening in the needle. This creates a stopper at the back of the hollow angled bevel that once introduced into the tissue not only adds no benefit to steering but begins to act as an impediment to the progress of the needle into the patient due to its square end configuration.

Plugged or unplugged, steering the needle is difficult and non precise as the open end of the angled bevel is hollow and does not provide a surface for the tissue to deflect off of.

None of these plugs or fillings contribute to reducing the initial penetration of the skin and its subsequent motion through the tissue.

The above-described synthetic plug is formed inside the needle causing it to “stick” to the needle requiring the user to “pop” it loose. Once popped loose it is beyond the needle bore and cannot contain the seeds it was holding back. This sudden release of pressure as the cannula is pulled back contributes to needle movement which can result in inaccurate placement of the seeds and spacers.

### SUMMARY

An apparatus for implanting an element within a tissue includes a cannula having a sharpened tip at a beveled distal end, and a stylet. The stylet is configured to engage the element within a hollow bore of the cannula, so that the element is pushed through the hollow bore out of the beveled distal end onto the tissue when the cannula is moved relative to the stylet along an axis.

The apparatus further includes a bevel-shaped member insertable within the beveled distal end of the cannula. The bevel angle of the insertable member substantially matches a bevel angle of the cannula distal end. The insertable member has an outer surface that supports the tissue to prevent the tissue from prolapsing into a hollow interior of the beveled distal end while the sharpened tip penetrates the tissue.

The apparatus further includes a protruding structure extending from at least a portion of the outer surface of the member. The protruding structure is configured to frictionally hold the member in place along an interior surface of the hollow bore, while the cannula is moved relative to the stylet and the sharpened tip penetrates the tissue.

A bevel-shaped insert member is provided for a needle assembly that is configured to implant an element within a tissue. The needle assembly includes a cannula having a sharpened tip at a beveled distal end, and a stylet positionable within a hollow bore of the cannula. The insert member has a bevel angle that substantially matches a bevel angle of a beveled distal end of the cannula. The size of the insert member allows the member to substantially fill up the hollow interior of the beveled distal end of the cannula, when the insert member is inserted within the beveled distal end. The insert member has an outer surface that provides support to the tissue to prevent the tissue from prolapsing into a hollow interior of the beveled distal end, when the sharpened tip penetrates the tissue as a result of a motion of the cannula relative to the stylet.

A method of implanting an element within a tissue includes positioning the element within a hollow bore of a cannula having a sharpened tip at a beveled distal end thereof. The method further includes engaging the element with a stylet that is positioned within the hollow bore of the cannula. The method further includes inserting within a hollow interior of the beveled distal end of the cannula a bevel-shaped insert member having a protruding structure extending from at least a portion of an outer surface of the member. The method further includes causing the cannula to move relative to the stylet along an axis, so that the sharpened tip penetrates the tissue and the element is pushed through the hollow bore out of the beveled distal end onto the tissue, and so that the insert member is frictionally held in place along an interior surface of the hollow bore during the motion of the cannula to reduce a penetration force of the sharpened tip as the tip penetrates the tissue.

An apparatus for implanting an element within a tissue includes a cannula having a sharpened tip at a beveled distal end, and a bevel-shaped insert member. The insert member is insertable within the beveled distal end of the cannula. The bevel angle of the insertable member substantially matches a bevel angle of the cannula distal end. The insert member has an outer surface that supports the tissue to prevent the tissue from prolapsing into a hollow interior of the beveled distal end while the sharpened tip penetrates the tissue.

The stylet is configured to engage the element and the insert member within a hollow bore of the cannula, so that the element and the insert member are pushed through the hollow bore out of the beveled distal end onto the tissue when the cannula is moved relative to the stylet along an axis.

The apparatus further includes a protruding structure extending from at least a portion of the outer surface of the element. The protruding structure is configured to frictionally hold the element in place along an interior surface of the hollow bore, while the cannula is moved and the sharpened tip penetrates the tissue.

### BRIEF DESCRIPTION OF THE DRAWINGS

FIG. 1 illustrates a needle assembly for implanting a therapeutic element within a tissue, in accordance with one embodiment of the present disclosure.

FIGS. 2A-2C illustrate a bevel-shaped insert member in accordance with one embodiment of the present disclosure, located at different positions relative to a beveled distal end of a cannula in the needle assembly illustrated in FIG. 1.

### DETAILED DESCRIPTION

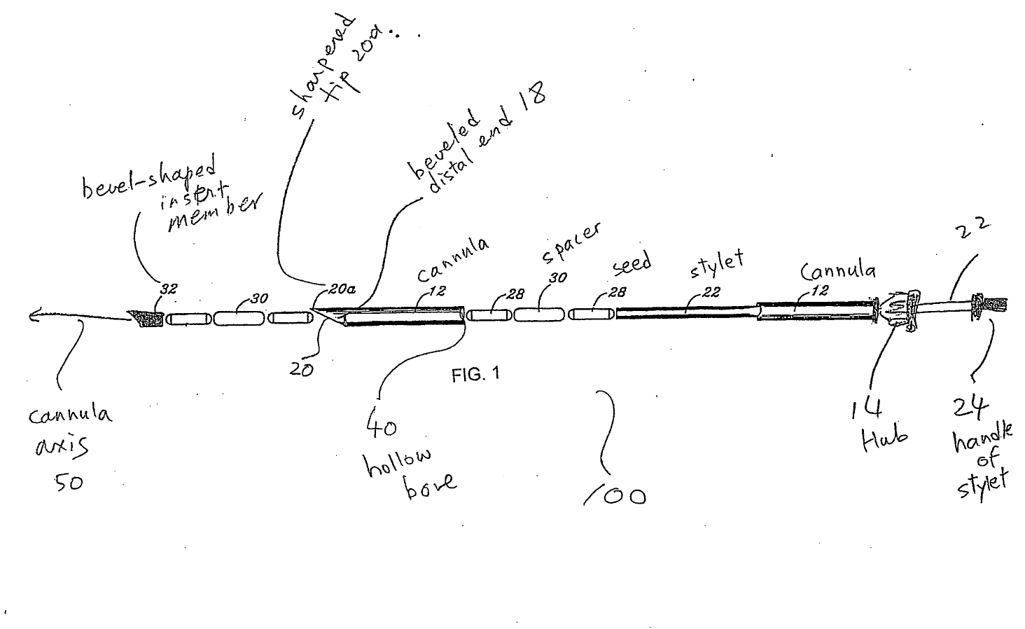

FIG. 1 illustrates an apparatus 100 (a needle assembly) for implanting a therapeutic element within a tissue, in accordance with one embodiment of the present disclosure. While the present illustration pertains to implantation of a therapeutic element, it should be understood that the element could be a therapeutic element, a diagnostic element or any other element that may be introduced via a needle assembly. In overview, the needle assembly 100 includes: a cannula 12 having a beveled distal end 18; a wire stylet 22; and a bevel-shaped insert member 32 insertable within the beveled distal end 18.

The cannula 12 has a tubular body 16 broken away in FIG. 1 to show its contents. The distal end 18 of the cannula 12 is shown as being beveled off at 20, to provide a point or sharpened tip 20a. The cannula 12 may have a hub 14 formed with gripping surfaces 14a. The cannula 12 may define a hollow bore 40 along a cannula axis 50.

The present illustration is described in terms of a needle assembly. It should be understood that the needle used with this type of assembly may be any type of needle that is used to introduce an element into the body. Such needles may include, but are not limited to, anesthesia needles, butterfly-type needles, standard hypodermic needles, spinal needles, marker needles, cardiology needles, radiology needles, introducer needles and insulin needles.

The wire stylet 22 is movably disposed within a hollow bore 40 of the cannula 12. The wire stylet 22 may have an handle 24 it its proximate end. The distal end of the stylet comprises an engagement surface 26. Also disposed within the cannula is a line of therapeutic elements, for instance, radioactive seeds 28. The seeds 28 may alternate with spacers 30 of cylindrical shape and made of a biocompatible and biodegradable material such as catgut.

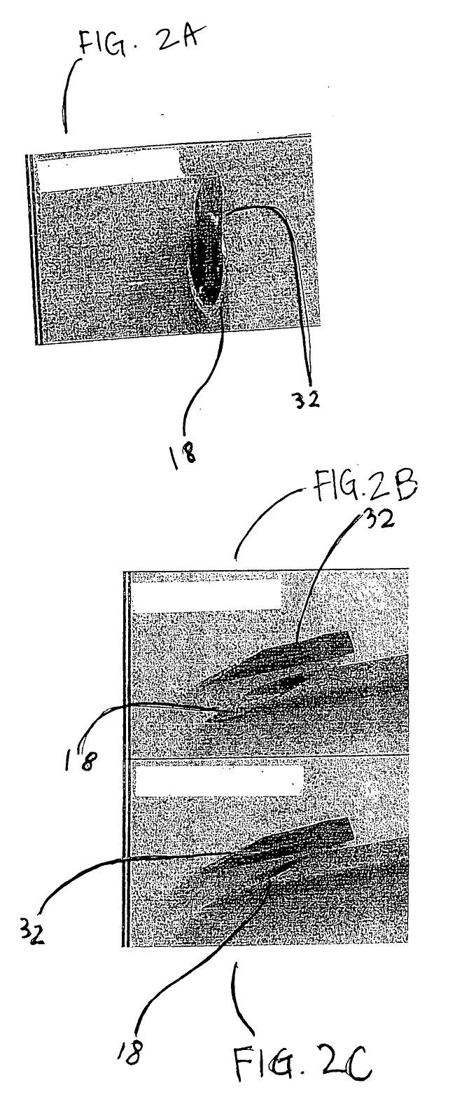

A bevel-shaped insert member 32 is disposed at the distal end 18 of the cannula 12, and is insertable therewithin. The bevel-shaped insert member 32 has a bevel angle (shown in FIG. 2 below) that matches the bevel angle of the distal end 18 of the cannula 12, thus providing a surface for the tissue to deflect off of. This greatly enhances the steerability of the needle, as explained in more detail in conjunction with FIG. 2.

The material of the insert member 32 may be biocompatible and biodegradable. It may be formed, for instance, of processed collagen (catgut), Nylon or various other organic substances. A preferred material may be polyglactin acid (PGA) available under the trademark POLYGLACTIN 910. The bevel-shaped insert member 32 may be forced outward as the cannula 12 is drawn backward on the stylet 22.

In operation, the needle assembly 100 shown in FIG. 1 may be inserted in the tissue of the body to be treated, distal end first. When the insertion is to the desired depth, the stylet 22 may be held firmly, and the cannula 12 may be drawn back toward the handle 24 of the stylet, causing the insert member 32 to give way from its initial position and deposit in the tissue the line of seeds and spacers. This motion of the cannula relative to the stylet leaves the seeds in the exact desired position in the body.

FIGS. 2A-2C illustrate a bevel-shaped insert member 32 in accordance with one embodiment of the present disclosure, located at different positions relative to a beveled distal end 18 of a cannula 12 in the needle assembly 100 illustrated in FIG. 1.

Because the tip of the cannula 12 is beveled or angled, the needle penetrates the tissue at an angle. The physician may try to compensate for this angled insertion, by rotating the needle as it is inserted to make it go to the proper place. This may be referred to as “steering.”

The needle assembly of the present disclosure may include a beveled or angled biocompatible insert 32 placed at the distal beveled tip of the needle. The angle of the insert matches the inherent bevel angle of the needle, thus providing a surface for the tissue to deflect off of, and greatly enhancing the steerabliity of the needle.

The beveled insert member 32 may be a suture plug that is molded to fit into the needle and conform to the beveled end and held in place by friction of protruberances (“wings”) posterior to the cutting heel. The insert member 32 may be made by molding to the beveled shape of needle, then applying hot bars to the plug which effectively squeeze out protruberances behind the bar.

This angled insert member 32 may be made of a variety of materials including absorbable or non-absorbable suture materials either in a braided or monofilament configuration or molded biocompatible polymers. In one form, one or more radioactive seeds may be implanted within the insert member 32.

These insert members 32 or plugs can be pre-made. The physician may thus put them in the hollow bore of the cannula, then put them in the strand of seeds and spacers behind them. These insert members 32 are a significant improvement over bone wax for steering, because they do not get pushed back up the needle.

The beveled or angled insert member 32 assists in reducing penetration force of the needle tip as it enters the tissue, by preventing the tissue from prolapsing into the hollow concaved pocket at the tip of the needle. The insert member holds the penetrated tissue up above the hollow end pocket of the needle, thus allowing the tissue to be cleanly cut by the sharpened edges of the angled bevel, and encourages the tissue to slide up the angled surface of the insert and allows the sharpened tip of the needle to pass on through the skin and into the tissue. The angled surface of the insert also holds the tissue up and allows it to flow over the top of the bevel during forward motion in the tissue thus minimizing the resistance of the entire needle to move smoothly forward without undue force or resistance.

The angled insert is held in place within the needle bore by a protruding structure extending from at least a portion of the outer surface of the member. The protruding structure is configured to frictionally hold the member in place along an interior surface of the hollow bore while the cannula is moved relative to the stylet and the sharpened tip penetrates the tissue.

In one embodiment, the protruding structure may include a plurality of wing-shaped protruberances, each protruberance extending from a respective portion of the outer surface of the member. In another embodiment, the protruding structure may be a substantially annular structure that extends peripherally around a circumference of the outer surface of the member.

In one form, the protruding structure may form “wings” at the proximal end of the insert that drag on the needle bore inner surface giving the insert smooth and constant drag as it is expelled from the needle eliminating the “pop” of the prior art synthetic plugs, thus improving the accuracy and location of the seeds as they exit the needle bore as the cannula is pulled back. These wings may avoid the popping out of the insert member, because they may keep up the friction as they are moving out of the needle.

In sum, a needle assembly for implanting an element (such as radioactive seeds) has been described, which has an beveled insert member at the tip of the needle that matches the needle's bevel angle, thus filling the hollow needle tip with a rigid material to improve needle steering and reducing needle penetration and subsequence movement within the tissue. The bevel-shaped insert member described in the present disclosure (1) reduces penetration force; (2) is less traumatic because reduces cutting by “cutting heel” because is bevel-shaped to match the needle rather than being a cylindrical plug; and (3) allows for better steering.

While certain embodiments have been described of an apparatus and method for pulsed deposition monitoring and control, it is to be understood that the concepts implicit in these embodiments may be used in other embodiments as well. The protection of this application is limited solely to the claims that now follow.

In these claims, reference to an element in the singular is not intended to mean “one and only one” unless specifically so stated, but rather “one or more.” All structural and functional equivalents to the elements of the various embodiments described throughout this disclosure that are known or later come to be known to those of ordinary skill in the art are expressly incorporated herein by reference, and are intended to be encompassed by the claims. Moreover, nothing disclosed herein is intended to be dedicated to the public, regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

What is claimed is:

1. An apparatus for implanting an element within a tissue, comprising:
a cannula having a sharpened tip at a beveled distal end;
a stylet configured to engage the element within a hollow bore of the cannula, so that the element is pushed through the hollow bore out of the beveled distal end onto the tissue when the cannula is moved relative to the stylet along an axis;
a bevel-shaped member insertable within the beveled distal end of the cannula and having a bevel angle that substantially matches a bevel angle of the distal end of the cannula, the member having an outer surface that supports the tissue to prevent the tissue from prolapsing into a hollow interior of the beveled distal end while the sharpened tip penetrates the tissue; and
a protruding structure extending from at least a portion of the outer surface of the member and configured to frictionally hold the member in place along an interior surface of the hollow bore while the cannula is moved and the sharpened tip penetrates the tissue.

2. The apparatus of claim 1, wherein the protruding structure comprises a plurality of wing-shaped protruberances, each protruberance extending from a respective portion of the outer surface of the member.

3. The apparatus of claim 1, wherein the protruding structure comprises a substantially annular structure that extends peripherally around a circumference of the outer surface of the member.

4. The apparatus of claim 1, wherein the insertable member has a size that allows the member to substantially fill up the hollow interior of the beveled distal end of the cannula, when inserted therewithin.

5. The apparatus of claim 1, wherein the insertable member comprises a biocompatible material.

6. The apparatus of claim 5, wherein the biocompatible material comprises at least one of:
an absorbable suture material;
a non-absorbable suture material; and
a molded biocompatible polymer.

7. The apparatus of claim 6, wherein the biocompatible material has at least one of:
a braided configuration; and
a monofilament configuration.

8. The apparatus of claim 1, wherein the element is a therapeutic element or a diagnostic element.

9. The apparatus of claim 1, wherein the element is a therapeutic element, and the therapeutic element comprises a radioactive seed.

10. The apparatus of claim 9, wherein the radioactive seed is implantable within the insertable member.

11. The apparatus of claim 1, wherein the element is a therapeutic element, and wherein the therapeutic element comprises a plurality of radioactive seeds, and wherein each of the radioactive seeds are separated from one another by one or more spacers.

12. The apparatus of claim 11, wherein the plurality of radioactive seeds are encapsulated within a biodegradable material.

13. The apparatus of claim 1, wherein the protruding structure is further configured to reduce a penetration force of the sharpened tip as the tip penetrates the tissue, by frictionally holding the member in place along the interior surface of the hollow bore during the motion of the cannula.

14. A bevel-shaped insert member for a needle assembly that is configured to implant an element within a tissue and that includes a cannula having a sharpened tip at a beveled distal end and a stylet positionable within a hollow bore of the cannula,
the insert member having a bevel angle that substantially matches a bevel angle of a beveled distal end of the cannula and having a size that allows the member to substantially fill up the hollow interior of the beveled distal end of the cannula when inserted therewithin,
the insert member having an outer surface that provides support to the tissue to prevent the tissue from prolapsing into a hollow interior of the beveled distal end when the sharpened tip penetrates the tissue as a result of a motion of the cannula relative to the stylet.

15. The insert member of claim 14, wherein the element is a therapeutic element or a diagnostic element.

16. A method of implanting an element within a tissue, comprising:
positioning the element within a hollow bore of a cannula having a sharpened tip at a beveled distal end thereof;
engaging the element with a stylet positioned within the hollow bore of the cannula;
inserting within a hollow interior of the beveled distal end of the cannula a bevel-shaped insert member having a protruding structure extending from at least a portion of an outer surface of the member;
causing the cannula to move relative to the stylet along an axis, so that the sharpened tip penetrates the tissue and the therapeutic element is pushed through the hollow bore out of the beveled distal end onto the tissue, and so that the insert member is frictionally held in place along an interior surface of the hollow bore during the motion of the cannula to reduce a penetration force of the sharpened tip as the tip penetrates the tissue.

17. The method of claim 16, wherein the element is a therapeutic element or a diagnostic element.

18. An apparatus for implanting an element within a tissue, comprising:
a cannula having a sharpened tip at a beveled distal end;
a bevel-shaped insert member insertable within the beveled distal end of the cannula and having a bevel angle that substantially matches a bevel angle of the distal end of the cannula, the member having an outer surface that supports the tissue to prevent the tissue from prolapsing into a hollow interior of the beveled distal end while the sharpened tip penetrates the tissue;
a stylet configured to engage the element within a hollow bore of the cannula, so that the element and the insert member is pushed through the hollow bore out of the beveled distal end onto the tissue when the cannula is moved relative to the stylet along an axis; and
a protruding structure extending from at least a portion of the outer surface of the element and configured to frictionally hold the member in place along an interior surface of the hollow bore while the cannula is moved and the sharpened tip penetrates the tissue.

19. The apparatus of claim 18, wherein the element is a therapeutic element or a diagnostic element.
